(19) United States
(12) Patent Application Publication
(10) Pub. No.: US 2026/0166075 A1
(43) Pub. Date: Jun. 18, 2026

(54) NANOZYME AND USE FOR TREATING NEURODEGENERATIVE DISEASE

(71) Applicants: The Johns Hopkins University, Baltimore, MD (US); Xuchang University, Xuchang (CN)

(72) Inventors: Xiaobo Mao, Baltimore, MD (US); Weiwei He, Xuchang (CN)

(21) Appl. No.: 19/127,030

(22) PCT Filed: Nov. 3, 2023
(86) PCT No.: PCT/US2023/078741

§ 371 Date: May 2, 2025

(30) Foreign Application Priority Data
Nov. 4, 2022 (CN) 202211378496.7

(51) Int. Cl. A61K 33/34; A61K 9/14; A61K 33/24; A61P 25/16; A61P 25/28

(52) U.S. Cl. CPC A61K 33/34; A61K 9/14; A61K 33/24; A61P 25/16; A61P 25/28

(57) ABSTRACT

The disclosure relates to pharmaceutical compositions including Ir/Cu nanoenzymes, methods of treatment for neurodegenerative disease or disorder, and kits including the compositions. The Ir/Cu nanoenzymes can suppress oxidative stress in neuronal cells and inhibit aggregation of alpha-synuclein.

The present application claims the benefit of Chinese Patent Application No. 202211378496.7, filed Nov. 4, 2022, which is incorporated herein by reference in its entirety.

### TECHNICAL FIELD

The disclosure relates to pharmaceutical compositions including Ir/Cu nanoenzymes, methods of treatment using the compositions, and kits including the compositions.

### BACKGROUND

Emerging evidence shows that oxidative stress can induce protein aggregation (e.g. α-synuclein, tau), and subsequent prion-like pathology propagation may be an essential driver in the pathogenesis of neurodegenerative diseases, including Parkinson's disease (PD) and Alzheimer's disease (AD). Therefore, compounds (e.g., small molecules, artificial enzymes or inorganic substance) that can suppress oxidative stress and inhibit the protein aggregation in neural cells may provide a potential candidate for the treatment of neurodegenerative diseases.

### SUMMARY

Provided are, inter alia, Ir-related nanozymes having strong scavenging ability on oxidative stress, use thereof for blocking the pathological protein aggregation, e.g., pathogenic prion-like α-synuclein and tau, and use for treating neurodegenerative diseases (e.g., Parkinson's disease (PD) and Alzheimer's disease (AD)).

In an aspect, provided is a pharmaceutical composition including an effective amount of one or more metallo-nanozymes and a pharmaceutically acceptable carrier. The nanozymes include one or more copper (Cu) components and/or one or more iridium (Ir) components.

Preferably, the metallo-nanozyme includes Cu, Ir, IrCu, Ir₃Cu, IrCu₃, or combinations thereof.

The metallo-nanozymes may be formed in particles or clusters. An average diameter of the metallo-nanozymes ranges from about 1 nm to 10 nm. Preferably, the average diameter of the metallo-nanozymes ranges from about 3 nm to 5 nm.

A suitably weight ratio between the Cu components and th [symbol] from 10:1 to about 1:10.

In a further aspect, a metallo-nanozyme is provided that comprises comprising one or more copper (Cu) components and/or one or more iridium (Ir) components. In aspects, the metallo-nanozyme suitably may comprises one or more copper (Cu) components and one or more iridium (Ir) components. In asepcts, the metallo-nanozyme comprises Cu, Ir, IrCu, Ir₃Cu, IrCu₃, or combinations thereof.

In aspects, a plurality of metallo-nanozymes is provided.

In aspects, the one or more the metallo-nanozyme may be formed in particles or clusters.

In aspects, an average diameter of one or more metallo-nanozyme ranges from about 1 nm to 10 nm. In aspects, the average diameter of one or more of the metallo-nanozymes ranges from about 3 nm to 5 nm.

In aspects, a weight ratio between the Cu components and the Ir components of a metallo-nanozyme (or a plurality of metallo-nanozymes) ranges from 10 to 1 to about 1:10.

In an aspect, provided is a method of treating a subject suffering from alpha-synucleinopathies or susceptible to alpha-synucleinopathies and the method includes administering to the subject the pharmaceutical composition or one or more of the metallo-nanozymes as described herein. In aspects, the subject is first identified as suffering from alpha-synucleinopathies and/or susceptible to alpha-synucleinopathies, and the pharmaceutical composition or one or more metallo-nanozymes is administered to the identified subject. Alpha-synucleinopathies for treatment in accordance with the present metallo-nanozymes, compositons and methods include without limitation Parkinson's disease (PD) (including idiopathic and inherited forms of Parkinson's disease), dementia with Lewy bodies (DLB), Diffuse Lewy Body Disease (DLBD), Lewy body variant of Alzheimer's disease (LBVAD), combined Alzheimer's and Parkinson's disease, multiple system atrophy (MSA), and neurodegeneration with brain iron accumulation type-1 (NBIA-1). In certain aspects, the alpha synucleinopathy is Parkinson's disease (PD).

In an aspect, the disclosure provides a method of treating or delaying the onset of proteinopathy and the method includes administering administering to a subject in need thereof the pharmaceutical composition one or more of the metallo-nanozymes as described herein. In aspects, the subject is identified as in need of treatment of a proteinpathy, and the pharmaceutical composition or one or more metallo-nanozymes is [symbol] identified subject.

In an aspect, the disclosure provides a method of inhibiting alpha-synuclein aggregation in cells of a subject with or at risk of alpha-synucleinopathies and the method includes administering to a subject in need thereof the pharmaceutical composition one or more of the metallo-nanozymes as described herein.

In an aspect, provided is a method of inhibiting the alpha-synuclein cell-to-cell transmission in a subject suffering from alpha-synucleinopathies or at risk of suffering from alpha-synucleinopathies and the method includes administering to a subject in need thereof the pharmaceutical composition one or more of the metallo-nanozymes as described herein.

In another aspect, the disclosure provides a method of treating a subject having or at risk of having an alpha-synucleinopathy and the method includes administering to a subject in need thereof the pharmaceutical composition one or more of the metallo-nanozymes as described herein.

In another aspect, the disclosure provides a method of treating or delaying the onset of proteinopathy and the method includes administering to a subject in need thereof the pharmaceutical composition or one or more of the metallo-nanozymes as described herein. In aspects, the subject is identified as in need of treatment of a proteinpathy, and the pharmaceutical composition or one or more metallo-nanozymes are administered to the identified subject.

In another aspect, provided is method of inhibiting alpha-synuclein aggregation in cells of a subject having or at risk of alpha-synucleinopathies and the method includes administering to a subject in need thereof the pharmaceutical composition or one or more of the metallo-nanozymes as described herein. In aspects, the the subject is identified as in need of inhibiting the alpha-synuclein cell-to-cell transmission, and the pharmaceutical composition or one or more metallo-nanozymes are administered to the identified subject.

According to many aspects of the disclosure, the subject has or is susceptible to Parkinson's disease or Alzheimer's disease.

In another aesepct, methods are provided for treating a subject suffering from or susceptible to Parkinson's disease, comprising administering to the subject a pharmaceutical composition or one or more metallo-nanozymes as disclosed herein. In aspects, the subject is identified as suffering from Parkinson's disease, and the pharma [symbol] one or more metallo-nanozymes is administered to the identified subject.

In another aspect, methods are provided for treating a subject suffering from or susceptible to Alzheimer's disease, comprising administering to the subject a pharmaceutical composition or one or more metallo-nanozymes as disclosed herein. In aspects, the subject is identified as suffering from Alzhemer's disease, and the pharmaceutical composition or one or more metallo-nanozymes are administered to the identified subject.

In a further aspect, methods are provided for treating a subject suffering from or susceptible to Parkinson's disease, dementia with Lewy bodies, Diffuse Lewy Body Disease, Lewy body variant of Alzheimer's disease, Alzheimer's Disease, combined Alzheimer's and Parkinson's disease, multiple system atrophy, and/or neurodegeneration with brain iron accumulation type-1, the method comprising administering to the subject a pharmaceutical composition or one or more metallo-nanozymes of any one of claims 1 through 15. In aspects, the subject is identified as suffering from Parkinson's disease, dementia with Lewy bodies, Diffuse Lewy Body Disease, Lewy body variant of Alzheimer's disease, Alzheimer's Disease, combined Alzheimer's and Parkinson's disease, multiple system atrophy, and/or neurodegeneration with brain iron accumulation type-1, and and the pharmaceutical composition or one or more metallo-nanozymes are administered to the identified subject.

In embodiments, the subject is a mammal. In some embodiments, the mammal is a human.

According to many aspects of the disclosure, aggregation of alpha-synuclein is inhibited in the subject by administering the metallo-nanozymes comprising one or more copper (Cu) components and/or one or more iridium (Ir) components. Alternatively, neuronal cells in the subject are treated with the metallo-nanozymes comprising one or more copper (Cu) components and/or one or more iridium (Ir) components.

Also provided is a pharmaceutical kit including the pharmaceutical composition or one or more metallo-nanozymes as described herein and instructions for using the pharmaceutical composition or one or more metallo-nanozymes.

Where applicable or not specifically disclaimed, any one of the embodiments described herein are contemplated to be able to combine with any other one or more embodiments, even though the embodiments are described under different aspects of the invention.

These and other embodiments are disclosed or are obvious [symbol] the following Detailed Description.

### BRIEF DESCRIPTION OF THE DRAWINGS

FIGS. 1A-1G show TEM (FIG. 1A) and HRTEM (FIG. 1B) images of Ir₃Cu; HAADF-STEM image of Ir₃Cu (FIG. 1C); EDS elemental mapping of Cu (FIG. 1D); Ir (FIG. 1E) and overlap of Ir₃Cu (FIG. 1F), and cross-section composition (FIG. 1G) line profiles of one particle.

FIGS. 2A-2E show formation of IrCu with controllable alloy composition. HRTEM images of Ir (FIG. 2A), IrCu (FIG. 2B) and IrCu3 (FIG. 2C) nanozyme, the size distribution (FIG. 2D) and XRD patterns (FIG. 2E) of Ir and IrCu with different Ir/Cu compositions.

FIGS. 3A-3I show the antioxidant capacity of IrCu nanozyme to scavenge RONS and free radicals. (FIG. 3A) scheme for IrCu nanozyme to mimic 3 redox enzymes (POD: peroxidase, SOD: superoxide dismutase, CAT: catalase) and scavenge free radicals. (FIG. 3B) The UV-vis spectra of TMB in the presence of H2O2 catalyzed by POD-like Ir₃Cu NAs. The POD-like (FIG. 3C), CAT-like (FIG. 3D), SOD-like activity (FIG. 3E), and the activity to scavenge ·OH (FIG. 3F), DPPH· free radicals (FIG. 3G) and ONOO— (FIG. 3H) of Ir and IrCu nanozyme with different compositions. (FIG. 3I) Radar map for comprehensive comparison of antioxidant activity of different NPs.

FIGS. 4A-4H shows the ROS scavenging effect of nanozymes. (FIG. 4A) Representative images of ROS (green) in BV2 cells treated with αSyn PFF and nanozymes. (FIG. 4B) Flow cytometric analysis of ROS in BV2 cells treated with αSyn PFF and nanozymes. (FIGS. 4C, 4D) Corresponding quantification of (FIG. 4C) ROS mean fluorescence intensity (MFI) and (FIG. 4D) the percentage of ROS⁺ cells. (FIG. 4E) Representative images and (FIG. 4F) corresponding quantification of ROS fluorescence in microglia treated with αSyn PFF and nanozymes. (FIG. 4G) Representative images and (FIG. 4H) corresponding quantification of ROS fluorescence in primary neurons treated with αSyn PFF and nanozymes.

FIGS. 5A-5H shows the effect of various nanozymes on αSyn aggregation of HEK-A53T-YFP cells. (FIG. 5A) Representative images and (FIG. 5B) YFP fluorescence profiles of HEK-A53T-YFP cells with (right) and without (left) αSyn PFF treatment. (FIG. 5C) Representative images displaying the expression of αSyn in HEK-A53T-YFP cells simultaneously treated with αSyn PFF and nanozymes of Ir, Ir₃Cu, IrCu, or IrCu₃. (FIG. 5D) Corresponding quantification of the percentage of pathologic cells with aggerated αSyn to total cells. (FIG. 5E) Representative images of HEK-A53T-YFP c [symbol] hours before nanozymes treatments. (FIG. 5F) Relative profiles of YFP fluorescence indicated with red arrows in FIG. 5E. (FIG. 5G) Representative images and (FIG. 5H) relative quantification of Live/Dead staining showing survival of HEK-A53T cells treated with nanozymes. The cells heated at the 45° C. for 5 min were used as living and dead cell control.

FIGS. 6A-6B show the effect of various nanozymes on αSyn pathology of the primary neurons. (FIG. 6A) Representative images of NeuN (green) and phospho S129 (pS129, red) in primary neurons treated with nanozymes and αSyn PFF. (FIG. 6B) Quantification of pS129 in primary neurons.

FIGS. 7A-7I shows the enrichment of nanozymes in the brain and the effect of nanozymes on the brain-original Parkinson's disease mice model. (FIG. 7A) IVIS imaging of mice and (FIG. 7B) corresponding quantification of Cy5.5 fluorescence in the brain at 72 hours after intranasal (i.n.) injection with Cy5.5 labeled Ir. (FIG. 7C) Representative images and (FIG. 7D) corresponding quantification of Cy5.5 fluorescence in brain at 72 hours after intravenous (i.v.) injection with Cy5.5 labeled Ir. (FIG. 7E) corresponding quantification of Cy5.5 fluorescence in tissues including brain, blood, heart, liver, lung, spleen, and kidney. (FIG. 7F) The images of pathological αSyn (pS129, red) signal in the cortex of αSyn PFF-intrastriatal injected mice with Ir administration and (FIG. 7G) the corresponding quantification of pS129 signal. (FIG. 7H) The images of microglia (IBA1, red) signal in the cortex and (FIG. 7I) the corresponding quantification of the IBA1 signal.

FIG. 8A-8N shows the enrichment of nanozymes in the gut and the effect of nanozymes on the peripheral-original Parkinson's disease mice model. (FIG. 8A) IVIS imaging and (FIG. 8B) corresponding quantification of Cy5.5 fluorescence in the gastrointestinal tracts at 72 hours after Cy5.5 labelled Ir (Cy5.5-Ir) oral gavage. (FIGS. 8C-8J) Ir treatment in A53T mice with αSyn PFF intra-gut injection. Respective images and corresponding quantification of (FIGS. 8C, 8D) ROS, (FIGS. 8E, 8F) IBA1, (FIGS. 8G, 8H) pS129, and (FIGS. 8I, 8J) TH signals in the gut of mice with vehicle or Ir oral-gavage administration. (FIGS. 8K-8N) Representative images and corresponding quantification of (FIGS. 8K, 8L) IBA1 and (FIGS. 8M, 8N) pS129 signals in the brainstem of mice with vehicle or Ir oral-gavage administration. Central canal (CC).

### DETAILED DESCRIPTION

The following detailed description, given by way of example, but not intended to limit the invention solely to the specific embodiments described, may best be understood in conjunction with the accompanying drawings.

The present invention relates to pre-reacted, beaded composite materials comprising a hydrogel and a nanostructure for use in methods for reconstruction of soft tissue. The invention also relates to a soft tissue device comprising beaded composite materials for cell and tissue delivery for cosmetic, reconstructive, and cellular therapies.

The invention also relates to composite materials that can recruit, capture, encapsulate, associate, and/or embed specific tissue constituents including but not limited to adipocytes, other mesenchymal cells, or mesenchymal stem cells. The invention further relates to composite materials that can recruit, capture, encapsulate, associate, and/or embed specific tissues including but not limited to adipose tissues. The invention also relates to methods for repairing or reconstructing a soft tissue injury using a composition comprising a scaffold complex (such as soft tissue device) comprising a biomaterial covalently linked to a biodegradable fiber. The invention in other aspects also relates to a method of fabricating a composition for use in soft tissue reconstruction where the composition comprises a hydrogel and a nanostructure disposed therein. The invention in particular aspects also relates to a method of fabricating a composition for use in cell and tissue delivery for cosmetic, reconstructive, and cellular therapies.

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

Although any methods and materials similar or equivalent to those described herein can also be used in the practice or testing of the present invention, the preferred methods and materials are now described. All publications mentioned herein are incorporated herein by reference to disclose and described the methods and/or materials ir [symbol] the publications are cited.

Unless defined otherwise, all technical and scientific terms used herein have the meaning commonly understood by a person skilled in the art to which this invention belongs. The following references, the entire disclosures of which are incorporated herein by reference, provide one of skill with a general definition of many of the terms (unless defined otherwise herein) used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, the Harper Collins Dictionary of Biology (1991). Generally, the procedures of molecular biology methods described or inherent herein and the like are common methods used in the art. Such standard techniques can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York).

The following terms may have meanings ascribed to them below, unless specified otherwise. However, it should be understood that other meanings that are known or understood by those having ordinary skill in the art are also possible, and within the scope of the present invention. All publications, patent applications, patents, and other references mentioned herein are incorporated by reference in their entirety. In the case of conflict, the present specification, including definitions, will control. In addition, the materials, methods, and examples are illustrative only and not intended to be limiting.

### Definitions

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art.

A term “nanozyme” as used herein refers to an organic or inorganic substance having a biological catalytic activity, like an enzyme, under a physiological or biological conditions. The nanozyme particularly features in its size, e.g., having an average diameter in nanometer range, e.g., less than about 990 nm, less than about 900 nm, less than th [symbol] about 700 nm, less than about 600 nm, less than about 500 nm, less than about 400 nm, less than about 300 nm, less than about 200 nm, less than about 100 nm, less than about 50 nm, less than about 30 nm, or less than about 10 nm, wherein the diameters are measured at the maximum distances of two points on the surface of each nanozyme. In certain embodiments, the nanozyme may be in a globular shape or particle, which may aggregate, or form a clustered structure from the particles. The “metallo-nanozyme” as used herein refers to a nanoenzyme whose enzyme-like catalytic activity is originated from inorganic metal components (e.g., metal elements, metalloid, metal alloy, or the like). In certain embodiments, the metallo-nanozyme may suppress or scavenge oxidative stress of reactive oxygen species in physiological condition of neural tissues or cells, e.g., central nervous system or peripheral nervous system.

Synucleinopathies (also called α-Synucleinopathies) as referred to herein include are neurodegenerative diseases characterized by the abnormal accumulation of aggregates of alpha-synuclein protein in neurons, nerve fibers or glial cells. They are characterized by degeneration of the dopaminergic system and other areas of the central nervous system. They manifest clinically with motor alterations, cognitive impairment, autonomous dysfunction and neuropathologically with the formation of alpha-synuclein aggregates, sometimes in the form of Lewy bodies (LBs). Synucleinopathies include Parkinson's disease (PD), dementia with Lewy bodies (DLB), Lewy body variant of Alzheimer's disease, combined Parkinson's disease (PD) and Alzheimer's disease (AD), and multiple system atrophy (MSA).

A therapeutic activity refers to the activity of an agent that is or may be useful in the prophylaxis or treatment of a disease. The screening system can be in vitro, cellular, animal or human. Agents can be described as having therapeutic activity notwithstanding that further testing may be required to establish actual prophylactic or therapeutic utility in treatment of a disease.

By “subject” is meant an organism to which the methods of the invention can be applied and/or to which the agents of the invention can be administered. A subject can be a mammal, including a human, or a mammalian organ or mammalian cells, including a human organ and/or human cells.

“Treating” or “treatment” covers the treatment of a disease-state in a mammal, and includes: (a) preventing the disease-state from occurring in a mammal, in particular, when such mammal is predisposed to the disease-state but has not yet be [symbol] (b) inhibiting the disease-state, e.g., arresting it development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc). In some embodiments, the treating may not include preventing the disease-state.

A pharmaceutical composition comprising one or more therapeutic agents (e.g. one or more metallo-nanozymes) may be administered to a subject and may comprise conventional pharmaceutically acceptable carriers, excipients, or diluents.

The term “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” or “pharmaceutically acceptable carrier” means an excipient or carrier that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

### Compositions

In an aspect, the disclosure provides a pharmaceutical composition including an effective amount of one or more metallo-nanozymes. The metallo-nanozymes may include one or more copper (Cu) components and/or one or more iridium (Ir) components. Also provided are the metallo-nanozymes.

The copper components include any form of material (e.g., elemental, crystal, compound, salt) including at least copper element. The iridium components include any form of material (e.g., elemental, crystal, compound, salt) including at l [symbol] certain embodiments, the Cu components and the Ir components may form a lattice structure (e.g., Cu and Ir) or crystal clusters.

In certain embodiments, the metallo-nanozymes are formed in particles or clusters. The size (e.g., diameter) of the particles or clusters may be measured as the distance between two points on the particles or clusters. In certain embodiments, an average diameter of the metallo-nanozymes ranges from about 1 nm to about 10 nm, from about 2 nm to about 8 nm, or from about 3 nm to about 5 nm, or from about 4 nm to 5 nm.

The metallo-nanozymes may have regular or irregular shape. For example, the metallo-nanozymes may have globular shape with difference radius. Alternatiely, the metallo-nanozymes may have irregular shapes without uniformity.

In certain embodiments, the Cu—Ir components include Cu, Ir, IrCu, Ir₃Cu, IrCu₃, or combinations thereof. In certain embodiments, the metallo-nanozymes include at least one selected from Ir and Ir₃Cu.

In certain embodiments of the metallo-nanozymes, a weight ratio between the Cu components and the Ir components ranges from about 10:1 to about 1:10, from about 1:1 to about 1:10, from about 1:3 to about 1:10, or from about 1:3 to about 1:5. In certain embodiments of the metallo-nanozymes, a weight ratio between the Cu components and the Ir components is about 1:10, about 1:9, about 1:8, about 1:7, about 1:6, about 1:5, about 1:4, about 1:3, about 1:2, or about 1:1. In certain embodiments of the metallo-nanozymes, a weight ratio between the Cu components and the Ir components is about 10:1, about 9:1, about 8:1, about 7:1, about 6:1, about 5:1, about 4:1, about 3:1, about 2:1, or about 1:1.

In certain embodiments of the metallo-nanozymes, an atom ratio between the Cu components and the Ir components ranges from about 10:1 to about 1:10, from about 1:1 to about 1:10, from about 1:3 to about 1:10, or from about 1:3 to about 1:5. In certain embodiments of the metallo-nanozymes, an atom ratio between the Cu components and the Ir components is about 1:10, about 1:9, about 1:8, about 1:7, about 1:6, about 1:5, about 1:4, about 1:3, about 1:2, or about 1:1. In certain embodiments of the metallo-nanozymes, a atom ratio between the Cu components and the Ir components is about 10:1, about 9:1, about 8:1, about 7:1, about 6:1, about 5:1, about 4:1, about 3:1, about 2:1, or about 1:1.

In certain embodiments, the one or more metallo-nanozymes do not include a metal oxide (e.g., CuO, CuO₂, Cu₂O₃, IrO, IrO₂, Ir₂O₃, or the like). Also, in certain embodiments, the one or more metallo-nanozymes do not include a metal nitride [symbol] like). Further, in certain embodiments, the one or more metallo-nanozymes do not include a metal carbide (e.g., CuC, IrC, or the like).

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Optimal dosages for a particular metal nanozyme can be readily determined by known dosing protocols. In certain therapies, a Ir/Cu agent may be administered to a subject in amounts of 0.1 μg/kg and 100 μg/kg body weight of the subject, as a daily dose, administered once or multiple times during a day.

In embodiments, the subject for treatment is a human, either a male or female. In certain aspects, the human will be an adult (at least 16, 18 or 21 years of age) or elderly (e.g. at least 65, 70 or 75 years of age).

The pharmaceutical compositions can take any suitable form (e.g., liquids, aerosols, solutions, inhalants, mists, sprays; or solids, powders, ointments, pastes, creams, lotions, gels, patches and the like) for administration by any desired route (e.g., pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by [symbol] by addition to sterile infusion fluids for intravenous infusion; or in the form of a lotion, cream, foam, patch, suspension, solution, or suppository for transdermal or transmucosal administration.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In embodiments, the pharmaceutical composition comprises an injectable form.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption.

Suitable and preferred metallo nanozymes including Cu/Ir [symbol] prepared.

### Methods of Use

In an aspect, the treatment methods includes administering to the subject the pharmaceutical composition as described herein.

In certain aspect, the methods include treating a subject suffering from alpha-synucleinopathies or susceptible to alpha-synucleinopathies.

In certain aspect, the methods include treating or delaying the onset of proteinopathy.

In certain aspect, the methods include inhibiting alpha-synuclein aggregation in cells of a subject with or at risk of alpha-synucleinopathies.

In certain aspect, the methods include inhibiting the alpha-synuclein cell-to-cell transmission in a subject suffering from alpha-synucleinopathies or at risk of suffering from alpha-synucleinopathies.

In certain aspect, the methods include treating a subject having or at risk of having an alpha-synucleinopathy.

In certain aspect, the methods include treating or delaying the onset of proteinopathy.

In certain aspect, the methods include inhibiting alpha-synuclein aggregation in cells of a subject having or at risk of alpha-synucleinopathies.

Preferably, the subject has or is susceptible to Parkinson's disease or Alzheimer's disease.

As discussed, the subject (e.g. human) for treatment may be suffering from (including identified by the medical professional are suffering from) Parkinson's disease, dementia with Lewy bodies, Diffuse Lewy Body Disease, Lewy body variant of Alzheimer's disease, Alzheimer's Disease, combined Alzheimer's and Parkinson's disease, multiple system atrophy, and/or neurodegeneration with brain iron accumulation type-1.

In embodiments, the subject is a mammal. In some embodiments, the mammal is a human. In some embodiments, the mammal is not a human.

Particularly, aggregation of alpha-synuclein is inhibited in the subject by administering the metallo-nanozymes comprising one or more copper (Cu) components and/or one or more iridium (Ir) components. Alternatively, neuronal cells in the subject are treated with the metallo-nanozymes comprising one or more copp [symbol] one or more iridium (Ir) components.

Further, the disclosure provides treatment kits including (a) the pharmaceutical composition that includes one or more nanozymes as described herein and (b) instructions for using the pharmaceutical composition. In embodiments, the composition in the kit is suitable for delivery (e.g., local injection or oral administration) to a subject. Further, the kit may include a suitable applicator, which may be pre-filled with the pharmaceutical compositin or provided empty.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention, one or more syringes, one or more applicators, or a sterile solution suitable for reconstituting a pharmaceutical composition of the present invention

Although particular examples and uses for the hydrogel/nanostructure composites of the invention have been described herein, such specific uses are not meant to be limiting. The hydrogel/nanostructure composites of the invention can be used for any application generally used for known hydrogels, and in particular, are useful for the repair and/or regeneration of soft tissue anywhere in the body.

### EXAMPLES

### Example 1: IrCu Nanozyme With RONS Scavenging Activity For Treatment of Parkinson Disease

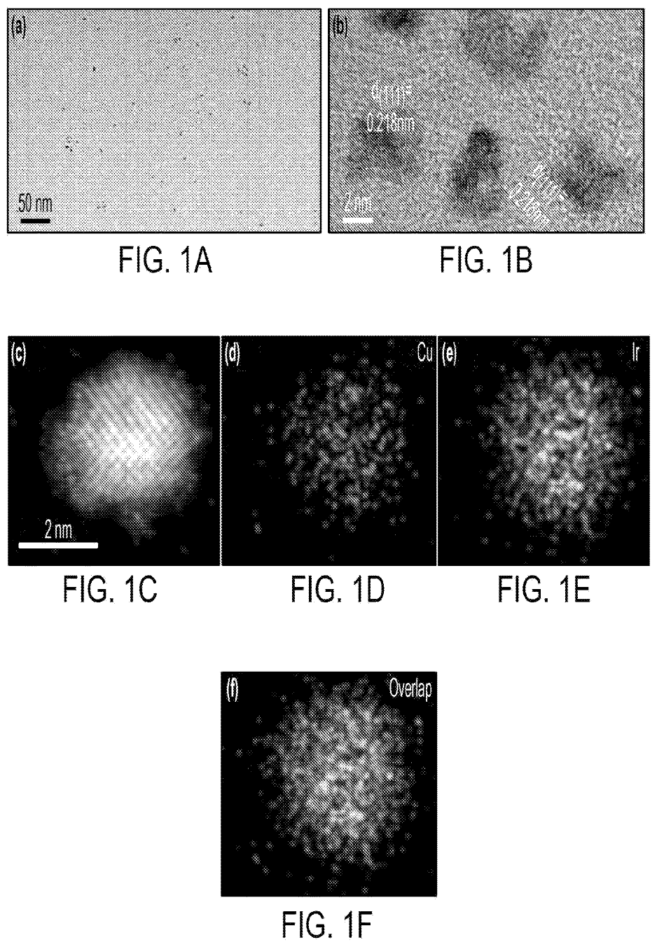

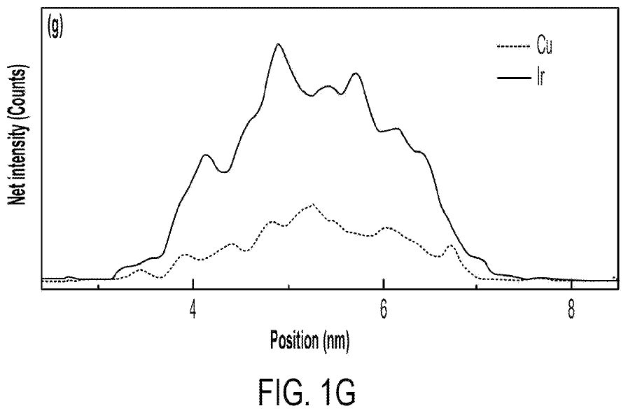

FIGS. 1A to 1G show the TEM images of IrCu nanozymes with Ir/Cu molar ratio of 3/1 (named Ir₃Cu). Ir₃Cu nanozymes are well dispersed in aqueous solution, have average diameter of 4.32±0.60 nm calculated from 200 random particles. The high-resolution TEM (HRTEM) further indicates the well-defined lattice planes in part of single particles. The calculated lattice spacing is 0.218 nm, which corresponds to the distance of (111) facet and falls between the values for Pt (0.222 nm) and Cu (0.208 nm). The EDS elemental mapping and line profile verify that the distribution of Ir and Cu in the particle is atomic mixture. These results indicate the alloy structure of Ir₃Cu NPs.

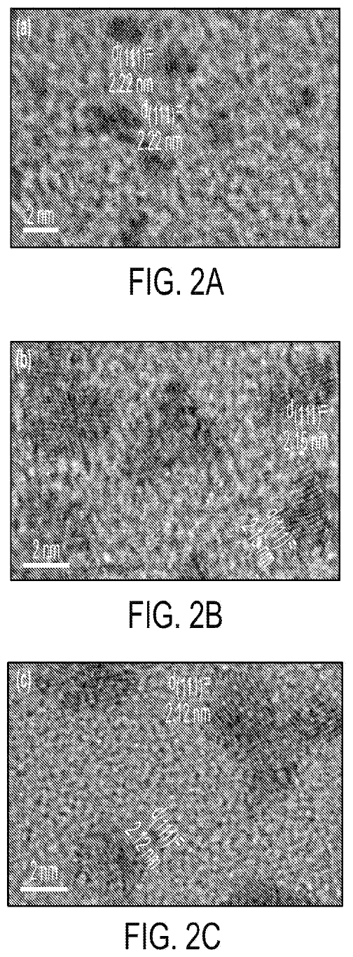

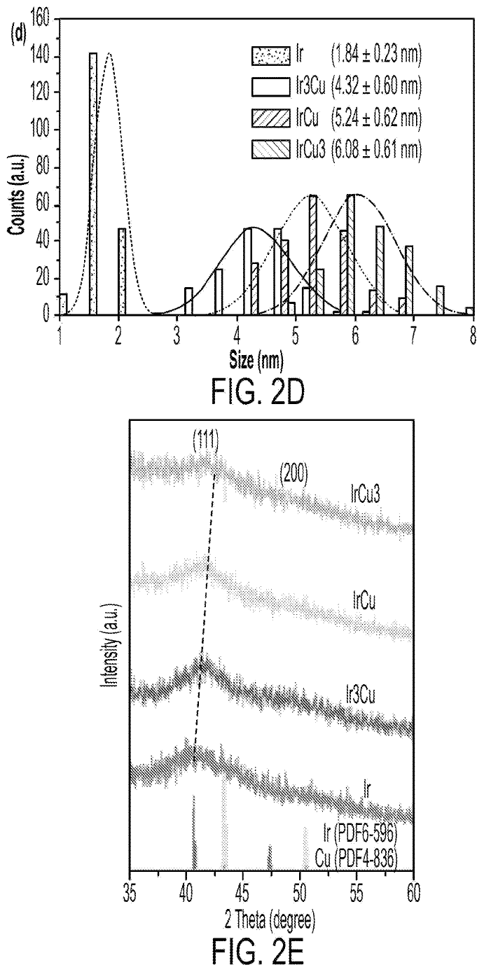

FIGS. 2A-2E show the TEM images of Ir, IrCu and IrCu₃. With increasing Cu percentage from 0, to 25%, 50%, 75%, it is found that, 1) the d-spacing of facet (111) gradually decrease, 2) the average size increases, and 3) the diffraction peak of (111) shifts to large 2 theta degree in the range between Ir (111) and Cu (111). These results not only demonstrate the formation of IrCu alloy, but also indicate that the alloy composition of IrCu can be finely adjusted by changing the feeding molar ratio of Ir³⁺/Cu²⁺.

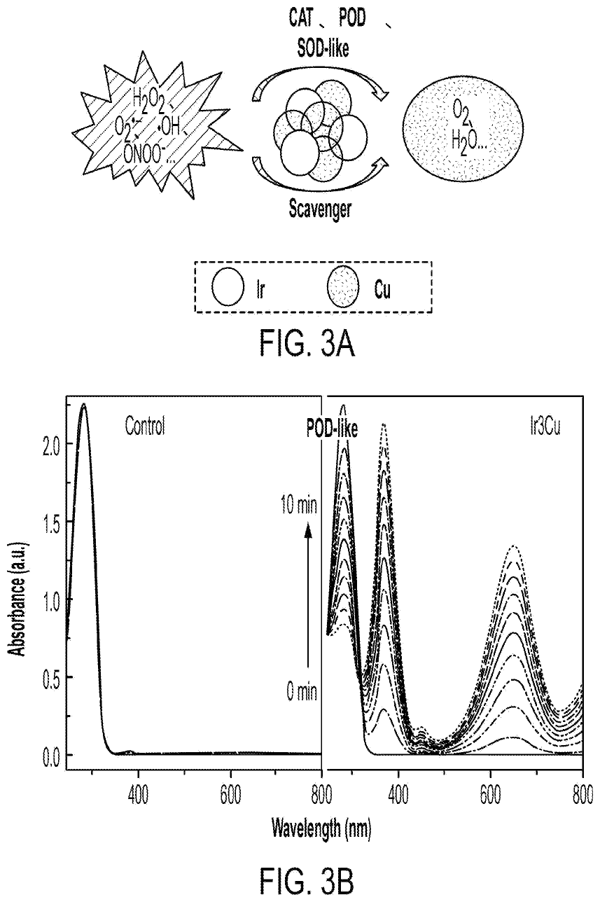

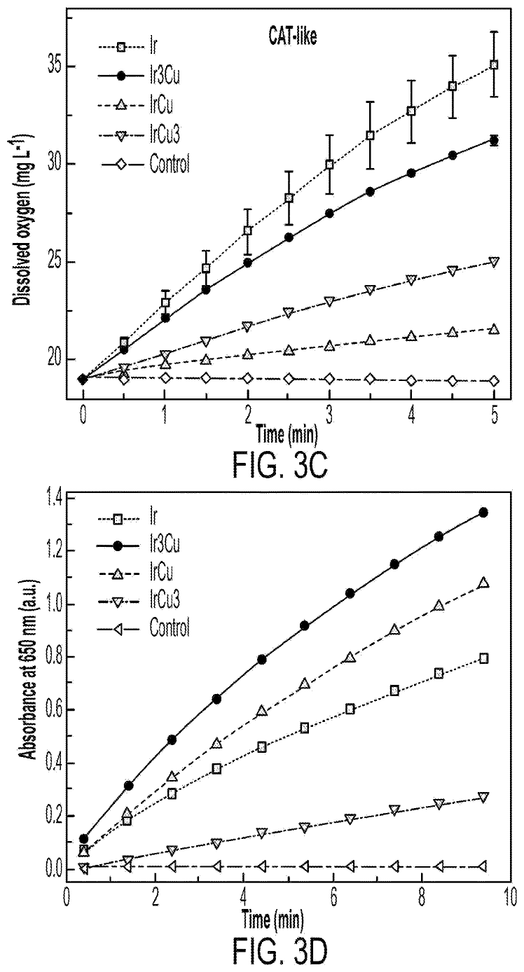

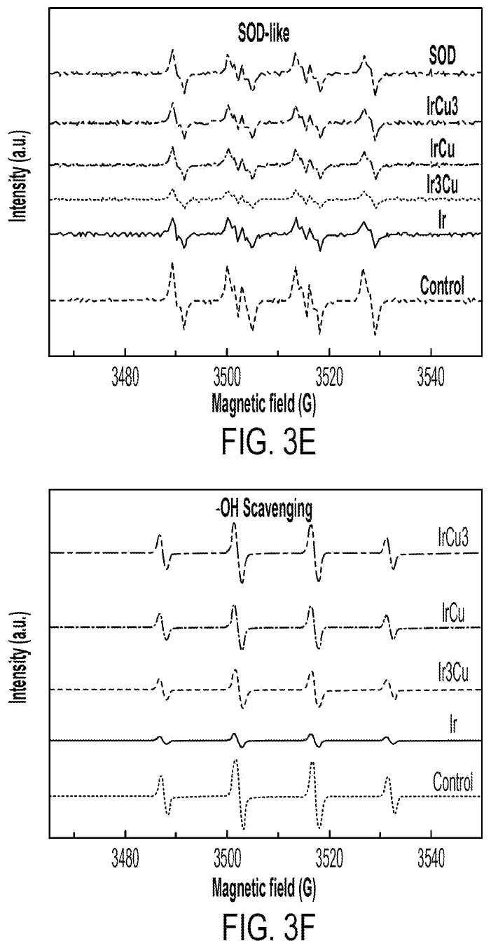

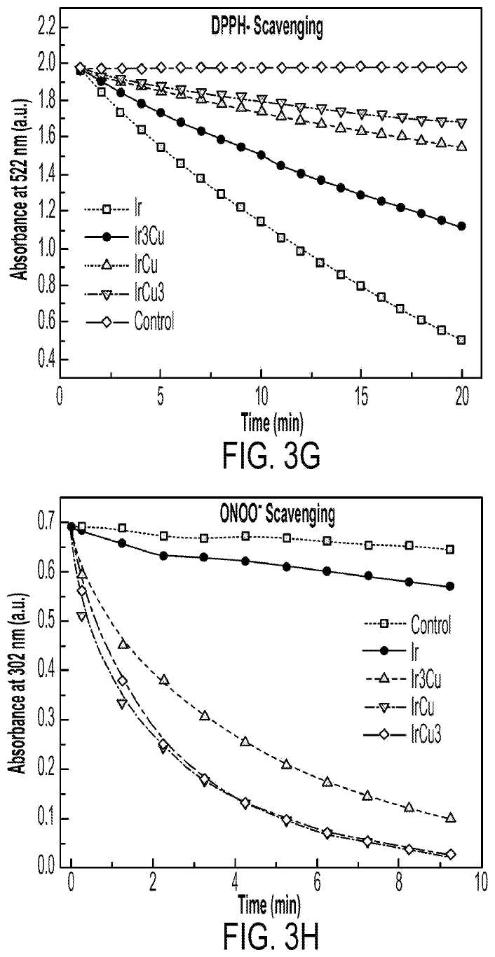

These results (FIGS. 3A-3I) demonstrate the multiple enzyme-like activity of IrCu nanozyme (POD, CAT and SOD-like activity) and ability to scavenge ·OH, DPPH· and ONOO—. It can eliminate ROS and RNS, suggesting superior antioxidant activity with broad spectrum. The antioxidant activities to scavenge RONS and free radicals of IrCu nanozymes are highly dependent on the alloy composition and nanozyme concentration.

Comprehensively, Ir and Ir₃Cu show the best antioxidant capacity.

### Nanozymes Have Ability to Scavenge Reactive Oxygen Species (ROS) Induced by α-Synuclein Preformed Fibril (α-Syn PFF) Stimulation In Vitro

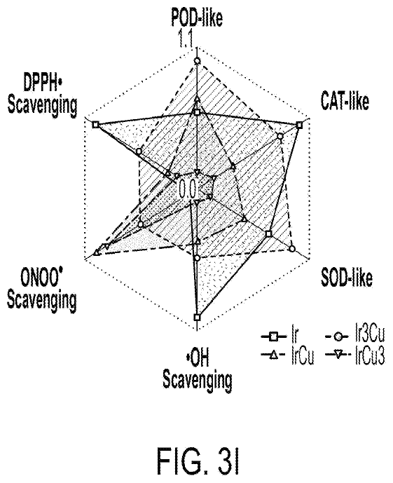

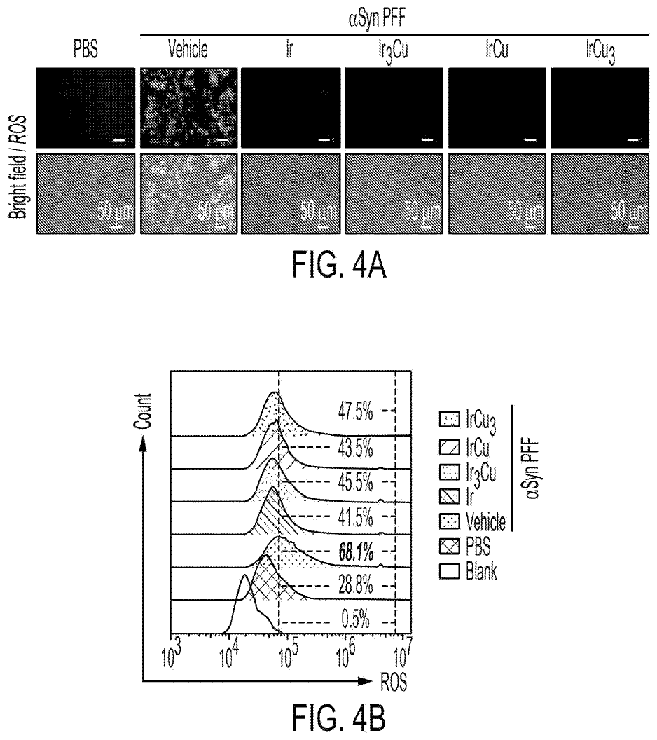

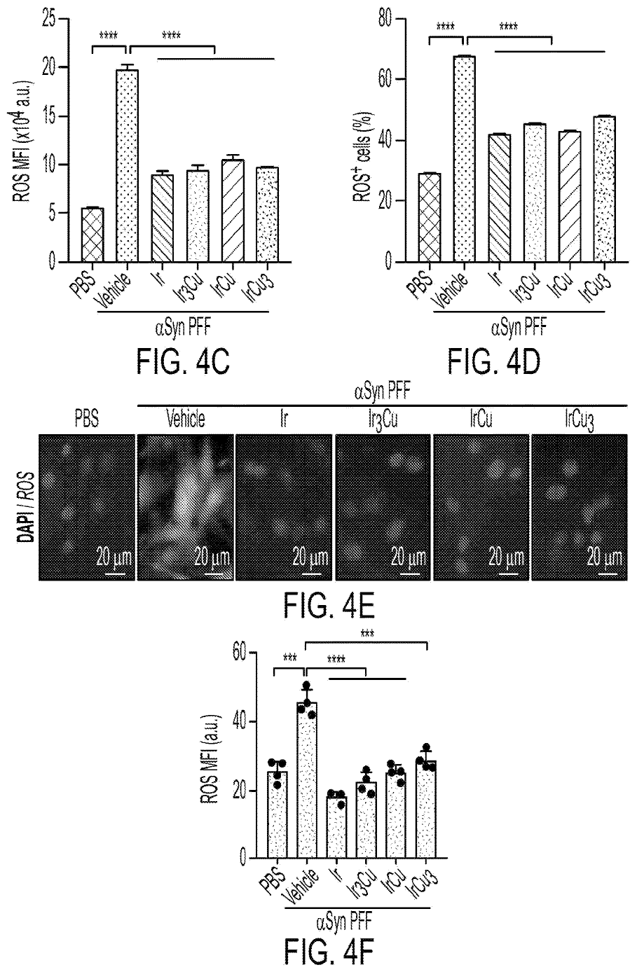

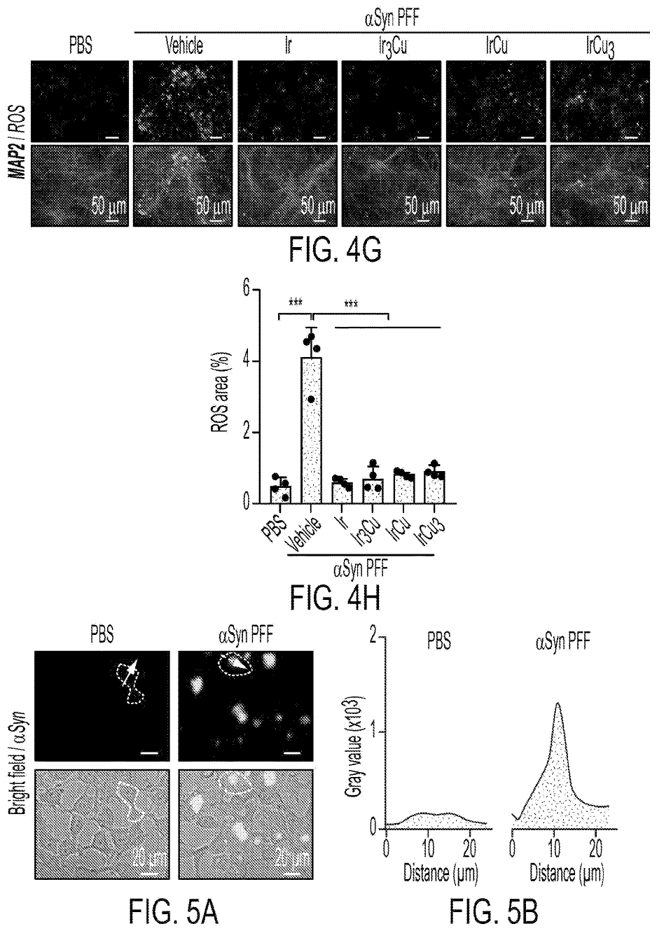

The BV2 cell, microglia, and primary neuron can produce ROS after α-Syn PFF stimulation. The nanozymes (Ir, Ir₃Cu, IrCu, or IrCu₃) treatment significantly reduced ROS accumulation in the cells (FIGS. 4A-4H).

Method: One day newborn pups from CD1 mice (Jackson Laboratory, Bar Harbor, ME, USA) were used for microglia culture. Recombinant mouse α-Syn protein was purified as previously published. α-Syn fibrils were prepared by agitation for 7 days. The fibrils were sonicated for 1 min (1 s on, 1 s off) at 30% amplitude (Branson Digital Sonifier, Branson Ultrasonics, Danbury, CT, USA) to obtain PFF. The microglia and [symbol] Syn PFF (20 μg/mL) and nanozymes of Ir, Ir₃Cu, IrCu, or IrCu₃ (1.25 μM) together. At 12 hours after treatment, the cells were added with CM-H2DCFDA (Thermo Fisher Scientific, Waltham, MA, USA) at 37° C. for 30 min. The ROS in the cells were detected by imaging or flow cytometry. To detect ROS in primary neuron, the neurons were treated with α-Syn PFF (5 μg/mL) and nanozymes of Ir, Ir₃Cu, IrCu, or IrCu₃ (1.25 μM) together for 2 days, and then using CM-H2DCFDA to detect the cellular ROS level.

### Nanozymes Have Ability to Reduce α-Syn Aggregation Without Cellular Toxicity

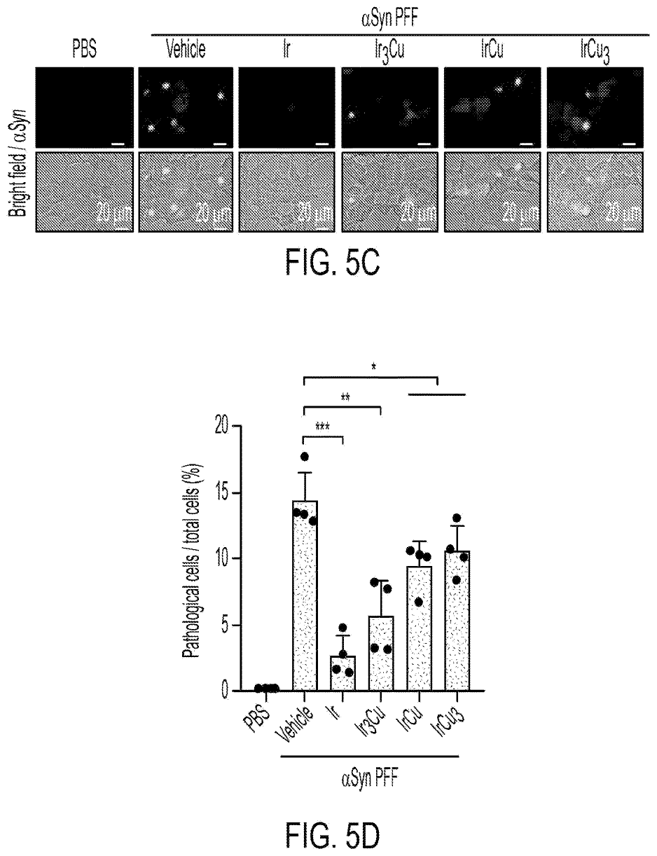

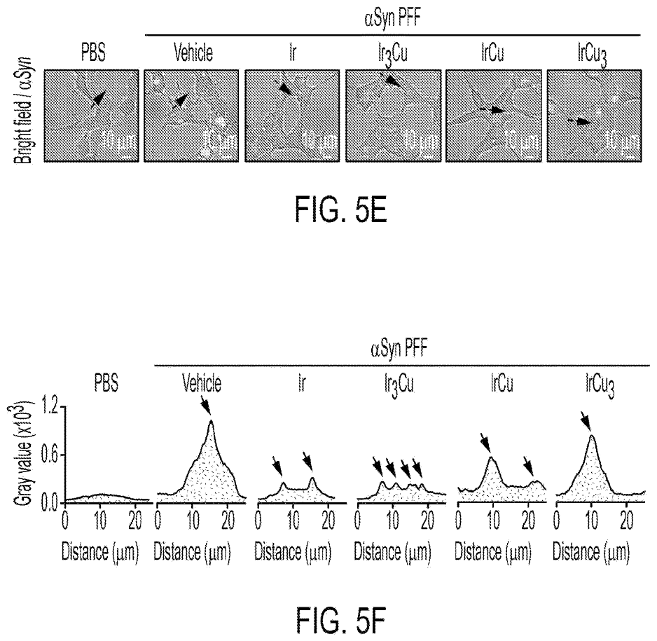

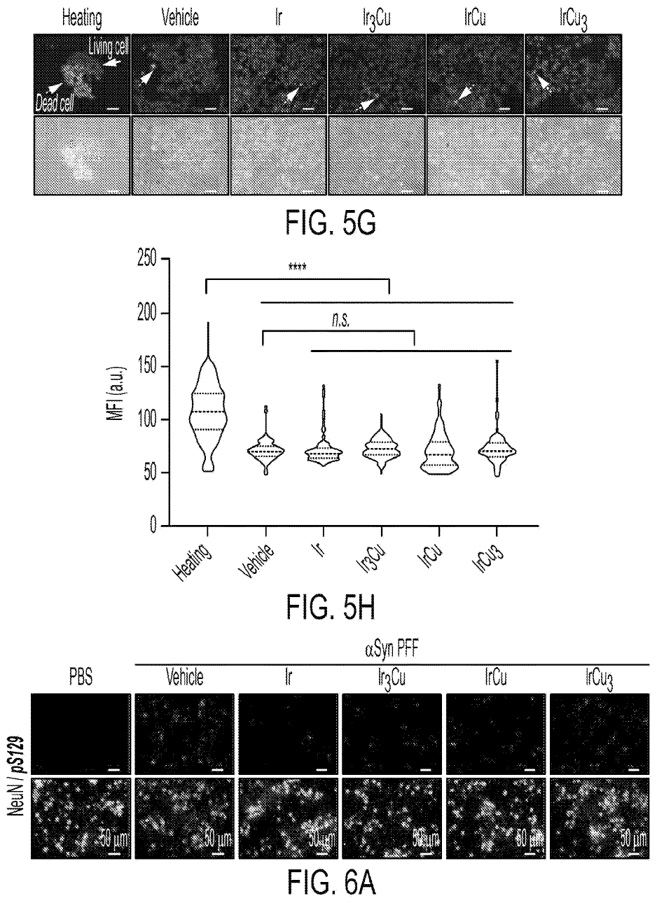

The HEK-A53T-YFP cells can express α-Syn with YFP fluorescence. Under normal condition, the expressed α-Syn is uniformly dispersed, consistent with the intracellular distribution of YFP fluorescence with low intensity in PBS group (FIG. 5A, B). After α-Syn PFF stimulation, intracellular α-Syn was aggregated, as evidenced by the clusters of YFP fluorescence in α-Syn PFF group (FIGS. 5A, 4B). To detect the effect of nanozymes to α-Syn aggeragation, α-Syn PFF and nanozymes were stimultaneously added into the HEK-A53T-YFP cells. Nanozymes treatment significantly reduced α-Syn aggregation as reflected in fewer clusters of YFP fluorescence compared with α-Syn PFF stimulation alone (FIGS. 5C, 5D). Additionally, Ir showed the best anti-aggregation capacity. To detect whether nanozymes have ability to degrade aggregates that have already formed, we stimulated the cells with α-Syn PFF 3 hours before nanozymes treatment. This interval is sufficient for α-Syn PFF to be taken up by the cells. At day 2 after nanozymes treatment, we imaged cellular α-Syn aggregates. In the group of α-Syn PFF treatment alone, we found that the profile of α-Syn aggregate was a single big peak (FIGS. 5E, 5F). However, after nanozymes treatment, the profile of α-Syn aggregate was scattered with small peaks in cells, especially in Ir and Ir₃Cu groups (FIGS. 5E, 5F). These results suggests that nanozymes have potential to degrade α-Syn aggregates. To test whether nanozymes have toxicity to the cells, we used Live/Dead staining to detect the cellular viability. Heating-induced death cells were used as positive control. None of the four nanozymes induced cell death compared with the control (FIGS. 5G, 5H).

Method: To detect the ability of nanozymes to reduce α-Syn aggregation, the HEK-A53T-YFP cells were treated by α-Syn PFF (5 μg/mL) alone or simultaneously treated with Ir, Ir₃Cu, IrCu, or IrCu₃ (125 μM). After 2 days, the cells were imaged to detect the α-Syn aggregates. To detect whether nanozymes have ability to degrade [symbol] already formed, we stimulated the cells with α-Syn PFF (5 μg/mL) 3 hours before nanozymes treatment (Ir, Ir₃Cu, IrCu, or IrCu₃ at the concentration of 125 μM). After 2 days, we taken images of the cells to detect the profiles of α-Syn aggregates. To test the cellular viability, the HEK-A53T-YFP cells were treated with Ir, Ir₃Cu, IrCu, and IrCu₃ (125 μM) for 12 hours. Then the cells were stained using LIVE/DEAD™ Fixable Aqua Dead Cell Stain Kit to show the live and dead cells. The cells heated in the water incubator at 45° C. for 5 min were used as dead cell control with brighter fluorescence.

### Nanozymes Have Ability to Reduce α-Syn Pathology in Primary Neuron Without Neurotoxicity

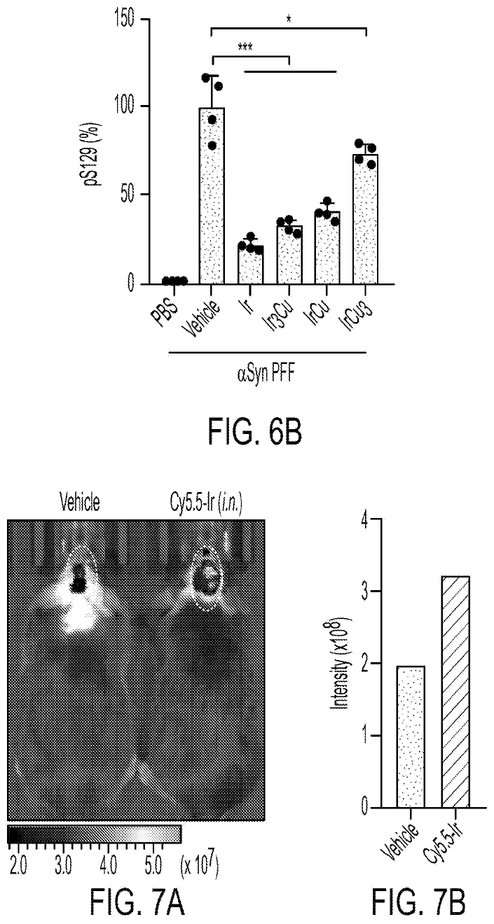

To detect whether nanozymes can reduce pathology induced by α-Syn PFF stimulation. The α-Syn PFF and nanozymes of Ir, Ir₃Cu, IrCu, or IrCu₃ were treated into primary neuron together. As the result, the pS129 signal, a typical pathological marker of pathology in Parkinson's disease was significantly reduced in the groups with nanozymes treatment (FIGS. 6A, 6B). Additionally, nanozymes treatment have no neurotoxicity as evidence by the number of NeuN (neuronal nuclei) staining and have no significant change compared to control (FIGS. 6C, 6D).

Method: Embryonic 15.5 days pups from CD1 mice (Jackson Laboratory, Bar Harbor, ME, USA) were used for primary cortical neuron culture. The plates for neuron culture were coated with 0.2 mg/mL Poly-L-ornithine for 1 hour and then washed 3 times with autoclaved milli-Q water. Neurobasal Medium and B-27 Supplement (Thermo Fisher Scientific, Waltham, MA, USA) were used for neuron culture. Primary neurons at 7 days were treated with α-syn PFF (5 μg/mL) and Ir, Ir₃Cu, IrCu, or IrCu₃ (125 μM) together. At day 7 after treatment, the neurons were stained with anti-NeuN antibody (1:500, MAB377, Sigma-Aldrich, St Louis, MO, USA) and anti-Alpha-synuclein (pS129) antibody (1:1000, ab51253, Abcam, Cambridge, MA, USA). For neurotoxicity assay, the neurons were treated with nanozymes of Ir and Ir₃Cu (125 μM) and α-syn PFF (5 μg/mL). At day 14 after treatment, the neurons were stained by anti-NeuN antibody.

### Nanozymes Enrich in the Brain and Reduce α-Syn Pathology

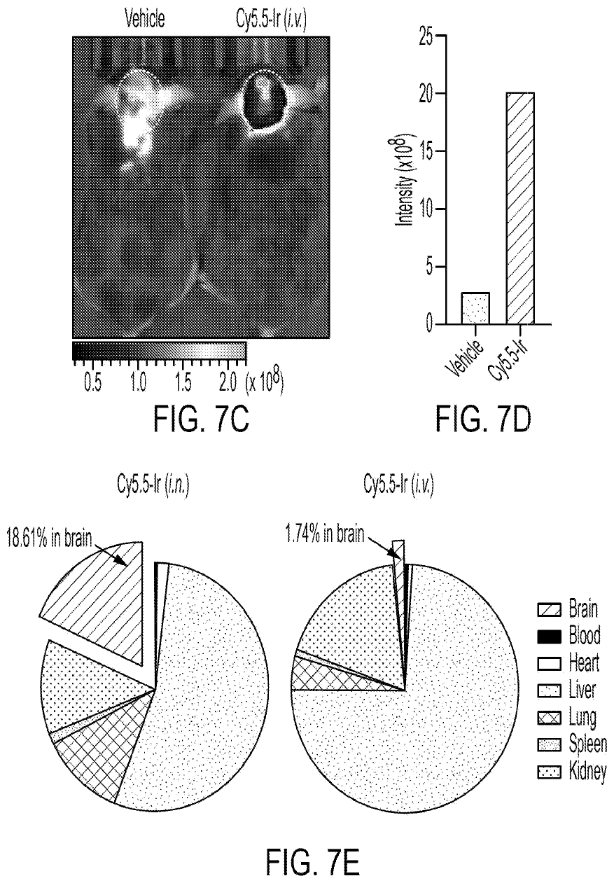

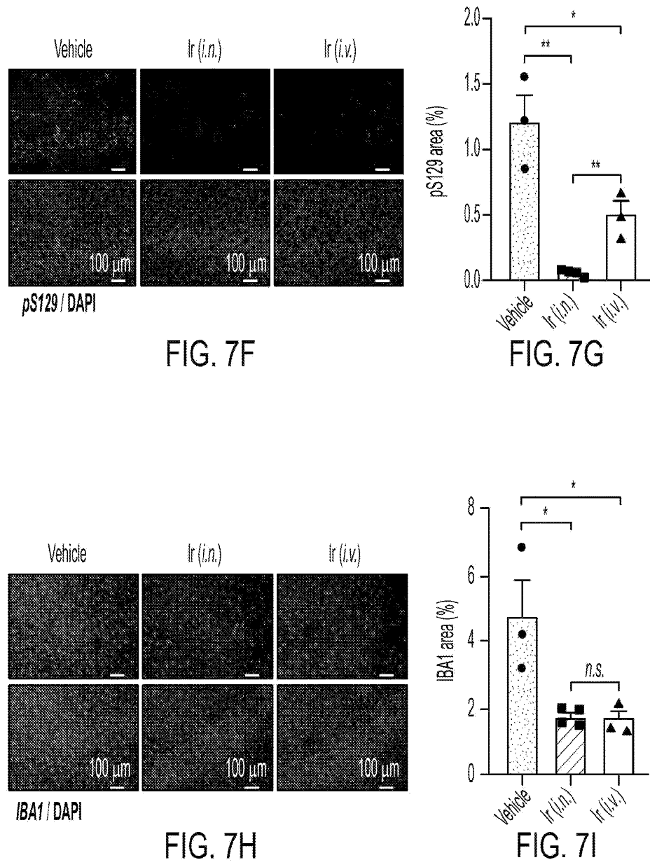

The nanozymes were labelled with Cy5.5 fluorescence and intranasally and intravenously administered into C57BL/6j mice. IVIS spectrum optical imaging was used to detect the distribution of nanozymes. The images and quantificati [symbol] accumulated into the brain after intranasal and intravenous injection (FIGS. 7A-7D). And we found that the brain enrichment effect of Ir nanozyme were 18.6% and 1.74%, respectively, through intranasal and intravenous injection Cy5.5 labelled Ir (FIG. 7E). Then we injected αSyn PFF into striatum to induce pathology. Two days after PFF treatment, the Ir nanozyme was weekly administered into the PFF-treated mice. After 9 weeks, the brains were collected to detect the pathology (pS129 signal). The results showed that Ir nanozyme treatment significantly reduced αSyn pathology (FIGS. 7F, 7G). And Intranasal injection showed higher ability to suppress αSyn pathology. Additionally, the activation of microglia labeled with ionized calcium binding adaptor molecule 1 (IBA1) was also obviously inhibited (FIGS. 7H, 7I). These results showed that nanozyme Ir treatment can suppress the αSyn pathology and related inflammation.

Method: Ir nanozyme was labelled with Cy5.5 PEG Thiol (Nanocs, New York, NY, USA) through incubation at room temperature for 1 hour. After incubation, the mixture was washed with Milli-Q water and purified by centrifugation at 14000 rpm for 20 min. To detect the distribution of nanozymes in mice, the Cy5.5-labelled Ir was intranasally and intravenously administer into the mice. After 3 days, the mice were imaged through IVIS Spectrum system. And the organs (brain, heart, liver, spleen, lung, kidney) and blood were collected for imaging. To test the effect of Ir on αSyn pathology and related inflammation, we constructed a αSyn PFF injected mice model. Two-month-old C57BL/6J mice received a single injection of 5 μg α-Syn PFF in 2 μL at striatum (+2.0 mm medial-lateral; +0.2 mm antero-posterior; +2.6 mm dorsoventral from bregma) using a 5 μL Hamilton syringe with a 30-gauge needle attached to a digital stereotaxic apparatus. Two days after PFF treatment, 100 μL Ir at the concentration of 5 mM was injected weekly into mice through intranasal and intravenous administration for total 6 times. After 9 weeks, the mice brains were collected for immunofluorescence studies of pathology and IBA1 using anti-pS129 antibody and anti-IBA1 antibody ((1:1000, 019-19741, Fujifilm Wako).

### Example 2: The Effect of Ir Nanozyme on the Spreading of Pathologic α-Syn From the Gut to the Brain Models Parkinson's Disease

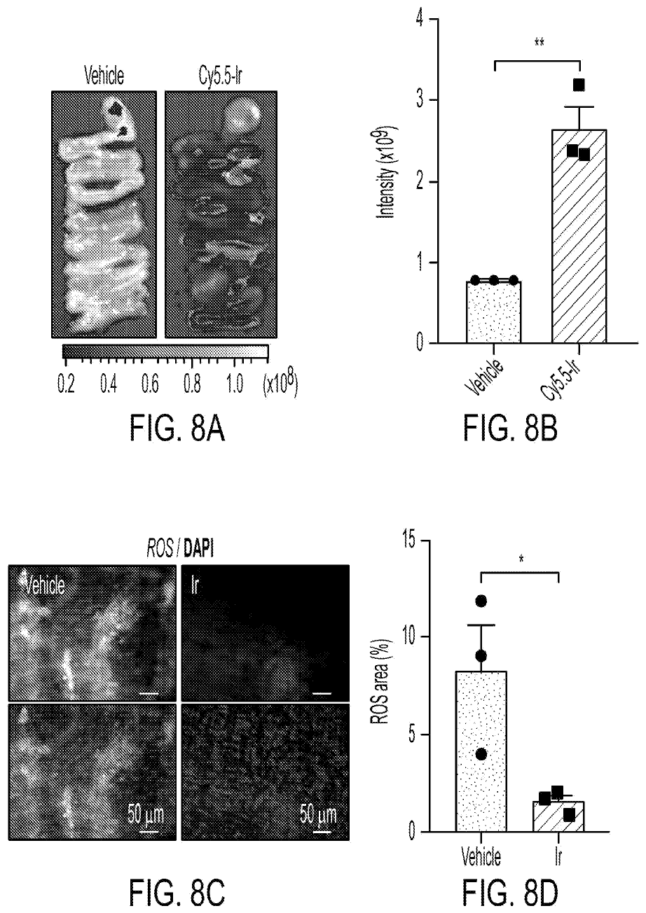

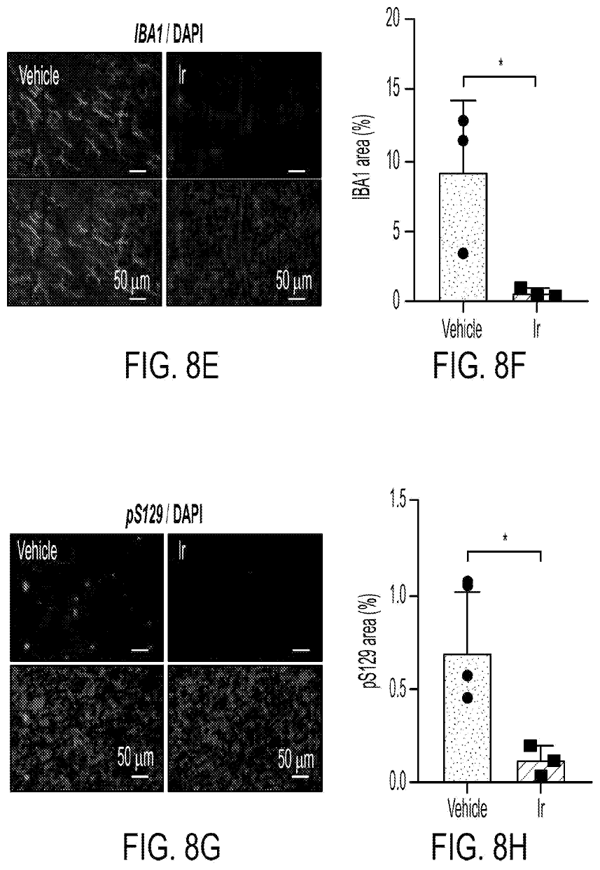

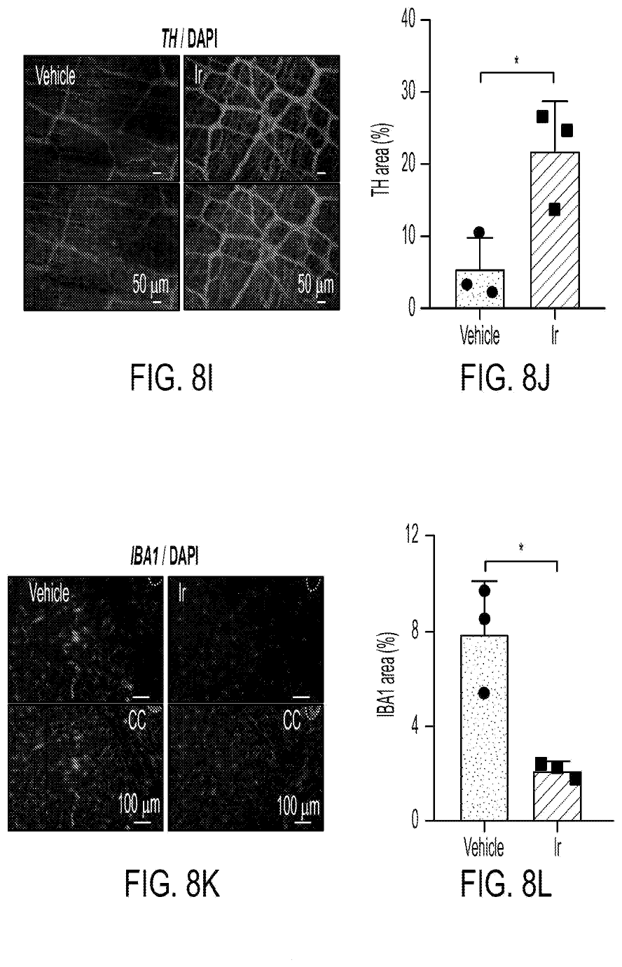

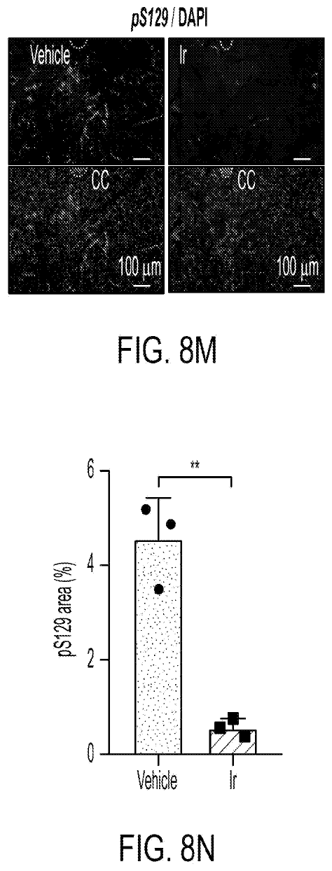

The α-syn pathology can spread from the gut via the vagus nerve into the brain. IVIS spectrum optical imaging was used to detect the distribution of nanozymes in the gut. The images and quantification showed that Ir was retented in the gut after 1 week of Cy 5.5-labelled Ir oral gavage administration (FIGS. 8A, 8B). Next, we w [symbol] injected Parkinson's Disease (PD) model to test whether Ir nanozyme have ability to relieve αSyn pathology spreading from gut into brain. The myenteric layer of the gut were separated for further detection. We found that αSyn PFF intra-gut injection induced ROS production in the gut and Ir oral-gavage siginificantly reduced ROS accumulation in the gut (FIGS. 8C, 8D). Meanwhile, the activation of enteric macrophage induced by αSyn PFF were suppressed by Ir treatment (FIGS. 8E, 8F). Further, αSyn pathology showed by the pS129 signal was reduced after Ir treatment (FIGS. 8E, 8F). The loss of tyrosine hydroxylase (TH) in the gut induced by αSyn pathology was recovered by Ir treatment (FIGS. 8I, 8J). We also found the microglia activation and αSyn pathology were inhibited in the brainstem after Ir treatment, as evidenced by the signal of IBA1 (FIGS. 8K, 8L) and pS129 (FIGS. 8M, 8N) were reduced in Ir group compare to vehicle group. These results suggested that Ir oral-gavage can remain in the gut and inhibit the inflammatory activation and further suppress the spreading of αSyn pathology from gut into brain.

Method: To detect the retention of nanozyme in the gut, the Cy5.5-labelled Ir was orally gavaged into the mice. After 7 days, the gut was collected and washed out of the contents and then imaged through the IVIS Spectrum system. Transgenic mice overexpress human αSyn with a PD-associated mutation (A53T) were used to construct the Syn PFF-gut-injected mice model. A53T mice at 3 months of age were anesthetized and put on a heat pad to keep body temperature. The αSyn PFF injection was conducted using a 10 μL Hamilton syringe into the wall of the pyloric stomach and intestine wall of the duodenum for a total of 25 μg αSyn PFF. Injections were made near the myenteric plexus. The mice were sutured and returned to normal housing conditions after injection. At 2 days after αSyn PFF injection, the mice were orally gavaged with Ir (200 μL, 10 mM) weekly. After 2 months, the mice were sacrificed to collect the brain and gut. The myenteric layer of the duodenum was isolated for immunostaining with CM-H2DCFDA, anti-IBA1 antibody, anti-pS129 antibody, and anti-tyrosine hydroxylase antibody (1:500, AB152, Sigma-Aldrich, St Louis, MO, USA). The brain sections were stained with anti-IBA1 antibody and anti-pS129 antibody.

### Equivalents

It is understood that the detailed examples and embodiments described herein are given by way of example for illustrative purposes only, and are in no way considered to be limiting to the invention. Various modifications or changes in ligh [symbol] to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

1. A pharmaceutical composition comprising an effective amount of one or more metallo-nanozymes comprising one or more copper (Cu) components and/or one or more iridium (Ir) components.

2. The pharmaceutical composition of claim 1 wherein one or more metallo-nanozymes comprising one or more copper (Cu) components and one or more iridium (Ir) components.

3. The pharmaceutical composition of claim 1, wherein the metallo-nanozyme comprises Cu, Ir, IrCu, Ir₃Cu, IrCu₃, or combinations thereof.

4. The pharmaceutical composition of claim 1, wherein the metallo-nanozymes are formed in particles or clusters.

5. The pharmaceutical composition of claim 1, wherein an average diameter of the metallo-nanozymes ranges from about 1 nm to 10 nm.

6. The pharmaceutical composition of claim 5 wherein the average diameter of the metallo-nanozymes ranges from about 3 nm to 5 nm.

7. The pharmaceutical composition of claim 1, wherein a weight ratio between the Cu components and the Ir components ranges from 10 to 1 to about 1:10.

8. A metallo-nanozyme comprising one or more copper (Cu) components and/or one or more iridium (Ir) components.

9. The metallo-nanozyme of claim 8 comprising one or more copper (Cu) components and one or more iridium (Ir) components.

10. The metallo-nanozyme of claim 8 wherein the metallo-nanozyme comprises Cu, Ir, IrCu, Ir₃Cu, IrCu₃, or combinations thereof.

11. A plurality of metallo-nanozymes of claim 1.

12-15. (canceled)

16. A method of treating a subject suffering from alpha-synucleinopathies or susceptible to alpha-synucleinopathies, comprising administering to the subject a pharmaceutical composition or one or more metallo-nanozymes of claim 1.

17-18. (canceled)

19. A method of treating or delaying the onset of proteinopathy, comprising administering a pharmaceutical composition or one or more metallo-nanozymes of claim 1 to a subject in need thereof.

20. (canceled)

21. A method of inhibiting alpha-synuclein aggregation in cells of a subject with or at risk of alpha-synucleinopathies, comprising administering a pharmaceutical composition or one or more metallo-nanozymes of claim 1 to a subject in need thereof.

22. A method of inhibiting the alpha-synuclein cell-to-cell transmission in a subject suffering from alpha-synucleinopathies or at risk of suffering from alpha-synucleinopathies, comprising administering a pharmaceutical composition or one or more metallo-nanozymes of claim 1 to a subject in need thereof.

23. (canceled)

24. A method of treating a subject having or at risk of having an alpha-synucleinopathy comprising administering a pharmaceutical composition or one or more metallo-nanozymes of claim 1.

25-28. (canceled)

29. A method of treating a subject suffering from or susceptible to Parkinson's disease, comprising administering to the subject a pharmaceutical composition or one or more metallo-nanozymes of claim 1.

30. (canceled)

31. A method of treating a subject suffering from or susceptible to Alzheimer's disease, comprising administering to the subject a pharmaceutical composition or one or more metallo-nanozymes of claim 1.

32. (canceled)

33. A method of treating a subject suffering from or susceptible to Parkinson's disease, dementia with Lewy bodies, Diffuse Lewy Body Disease, Lewy body variant of Alzheimer's disease, Alzheimer's Disease, combined Alzheimer's and Parkinson's disease, multiple system atrophy, and/or neurodegeneration with brain iron accumulation type-1, the method comprising:
administering to the subject a pharmaceutical composition or one or more metallo-nanozymes of claim 1.

34-38. (canceled)

39. A pharmaceutical kit comprising:
(a) a pharmaceutical composition one or more metallo-nanozymes of claim 1, and
(b) instructions for using the pharmaceutical composition one or more metallo-nanozymes.

40. (canceled)
